(19) United States
(12) Patent Application Publication
(10) Pub. No.: US 2015/0057350 A1
(43) Pub. Date: Feb. 26, 2015

(54) FIBROSIS BIOMARKERS AND METHODS OF USING SAME

(71) Applicant: ABBOTT LABORATORIES, ABBOTT PARK, IL (US)

(72) Inventors: SUZETTE PEREIRA, Westerville, OH (US); MENGHUA LUO, New Albany, OH (US); NEILE EDENS, Columbus, OH (US); GERARD DAVIS, Wauconda, IL (US); SUSAN GAWEL, LaGrange Park, IL (US); RAJ CHANDRAN, Evanston, IL (US)

(73) Assignee: ABBOTT LABORATORIES, ABBOTT PARK, IL (US)

(21) Appl. No.: 14/465,517

(22) Filed: Aug. 21, 2014

Related U.S. Application Data
(60) Provisional application No. 61/869,492, filed on Aug. 23, 2013.

(51) Int. Cl. A61K 31/19; G01N 33/573; A61K 31/191

(52) U.S. Cl. CPC A61K 31/19; A61K 31/191; G01N 33/573; G01N 2800/52
USPC 514/557; 435/23; 435/7.4; 506/9

(57) ABSTRACT

Methods and systems for using fibrosis biomarkers associated with a prolonged period of physical inactivity are provided. Also provided is a method of reducing the effect of prolonged physical inactivity on the development of fibrosis in a subject who is experiencing or is expected to experience prolonged physical inactivity in the near future by administering a therapeutically effective amount of a leucine metabolite (e.g., β-hydroxy-β-methylbutyric acid (HMB)) to the subject.

### FIELD

The general inventive concepts relate to biomarkers and methods of using the biomarkers. More particularly, the general inventive concepts relate to fibrosis biomarkers whose levels in blood change during prolonged or extended periods of physical inactivity, methods of using these biomarkers, and methods of reducing or attenuating the development of fibrosis associated with a prolonged period of physical inactivity in a subject.

### BACKGROUND

In various scenarios adults are required to undergo extended bed rest. For example, adults who are hospitalized due to illness, injury or surgery may be required to stay in bed for extended periods of time. Similarly, adults who are in a convalescent home, a rehabilitation center, or even at their own home may be bedridden or physically inactive over an extended period of time.

Extended bed rest or physical inactivity may lead to a number of complications such as low-grade inflammation (Hojbjerre, L., et al., Diabetes Care 34(10): 2265-2272, 2011), cardiovascular complications (Sonne, M. P., et al., Exp Physiol 96(10): 1000-1009, 2011), rapid muscle atrophy, and degenerative joint disease (Dittmer, D. K., et al., Can Fam Physician 39: 1428-1432, 1435-1437, 1993). Such complications may be related to the development of fibrosis in tissue due to dysregulated matrix metalloproteinase (MMP) activity. The development of fibrosis may in turn lead to the development of other conditions or complications such as arthritis, atherosclerosis, nephritis, tissue ulcers, aneurysms, and muscle atrophy.

### SUMMARY

The general inventive concepts relate to fibrosis biomarkers whose levels in blood change during prolonged or extended periods of physical inactivity, methods of using these biomarkers, and methods of reducing or attenuating the development of fibrosis associated with a prolonged period of physical inactivity in a subject. By way of example to illustrate various aspects of the general inventive concepts, several exemplary embodiments of methods and systems are provided herein.

In one exemplary embodiment, a method of reducing the effect of prolonged physical inactivity on the development of fibrosis in a subject who is experiencing or is expected to experience prolonged physical inactivity in the near future is provided. The method includes administering a therapeutically effective amount β-hydroxy-β-methylbutyric acid (HMB) to the subject.

In one exemplary embodiment, HMB is administered to the subject in an amount, in a manner, and for a time sufficient to enhance the increase in circulating levels of matrix metalloproteinase-1 (MMP-1) that occurs with prolonged physical inactivity in untreated control subjects.

In one exemplary embodiment, HMB is administered to the subject in an amount, in a manner, and for a time sufficient to attenuate the decrease in circulating levels of matrix metalloproteinase-3 (MMP-3) that occurs with prolonged physical inactivity in untreated control subjects.

In one exemplary embodiment, HMB is administered to the subject in an amount, in a manner, and for a time sufficient to attenuate the decrease in circulating levels of matrix metalloproteinase-10 (MMP-10) that occurs with prolonged physical inactivity in untreated control subjects.

In one exemplary embodiment, HMB is administered in an amount, in a manner, and for a time sufficient to keep a test level of at least one of MMP-3 and MMP-10 in a test biological sample taken from the subject 3 or more days after the subject has become physically inactive comparable to a baseline level of at least one of MMP-3 and MMP-10 in a baseline biological sample taken from the subject at the beginning of or prior to the prolonged physical inactivity.

In one exemplary embodiment, the subject who is experiencing or is expected to experience prolonged physical inactivity in the near future is a human subject. In one exemplary embodiment, the subject who is experiencing or is expected to experience prolonged physical inactivity in the near future is an adult human subject. In one exemplary embodiment, the subject who is experiencing or is expected to experience prolonged physical inactivity in the near future is an elderly human subject.

In one exemplary embodiment, a method of evaluating the efficacy of an intervention on the development of fibrosis in a subject who is experiencing or is expected to experience prolonged physical inactivity in the near future is provided. The method includes: (a) measuring levels of at least one biomarker selected from MMP-1, MMP-3, and MMP-10 in a baseline biological sample taken from the subject prior to or on the day the subject becomes physically inactive; (b) administering the intervention to the subject, wherein the intervention is first administered to the subject before or on the day the subject becomes physically inactive; (c) measuring levels of the at least one biomarker measured in (a) in a test biological sample taken from the subject 3 or more days after the subject becomes physically inactive; (d) calculating the difference between the levels of the at least one biomarker measured in the baseline biological sample and the at least one biomarker measured in the test biological sample; (e) comparing the differences calculated in (d) to corresponding control values based on the difference between levels of the at least one biomarker in comparable biological samples taken from untreated control subjects at comparable time points; and (f) determining that the intervention is efficacious when: (i) the difference for MMP-1 calculated in (d) is greater than the control value for MMP-1; (ii) the difference for MMP-3 calculated in (d) is smaller than the control value for MMP-3; (iii) the difference for MMP-10 calculated in (d) is smaller than the control value for MMP-10; or (iv) combinations of (i), (ii), and (iii).

In one exemplary embodiment, the baseline biological sample is a blood sample. In one exemplary embodiment, the test biological sample is a blood sample. In one exemplary embodiment, both the baseline biological sample and the test biological sample are blood samples. In one exemplary embodiment, the prolonged physical inactivity is bed rest. In one exemplary embodiment, the intervention is a leucine metabolite selected from HMB, alpha-ketoisocaproate (KIC), alpha-hydroxyisocaproate (HICA), and combinations thereof.

In one exemplary embodiment, a method of evaluating the efficacy of an intervention on the development of fibrosis in a subject who is experiencing or is expected to experience prolonged physical inactivity in the near future is provided. The method includes: (a) administering an intervention to the subject, wherein the intervention is first administered to the subject before or on the day the subject becomes physically inactive; (b) measuring the level of at least one biomarker selected from MMP-1, MMP-3, and MMP-10 in a test biological sample taken from the subject 3 or more days after the subject becomes physically inactive; (c) comparing the level measured in (b) to a baseline level of the at least one biomarker in a baseline biological sample taken from the subject before or on the day the subject becomes physically inactive; and (d) determining that the intervention is efficacious when the measured level of the at least one biomarker in the test biological sample is comparable to the baseline level of the at least one biomarker, respectively. In one exemplary embodiment, the at least one biomarker measured in (b) consists of MMP-1. In one exemplary embodiment, the at least one biomarker measured in (b) consists of MMP-3 and MMP-10. In one exemplary embodiment, the at least one biomarker measured in (b) consists of MMP-1, MMP-3, and MMP-10. In one exemplary embodiment, the intervention is a leucine metabolite selected from HMB, alpha-ketoisocaproate (KIC), alpha-hydroxyisocaproate (HICA), and combinations thereof. In one exemplary embodiment, the intervention is a nutritional composition comprising a leucine metabolite selected from HMB, alpha-ketoisocaproate (KIC), alpha-hydroxyisocaproate (HICA), and combinations thereof.

In one exemplary embodiment, a system for characterizing the efficacy of an intervention on the development of fibrosis associated with prolonged physical inactivity in a subject is provided. The system includes one or more sub-systems for: (a) identifying a subject undergoing physical inactivity or expected to undergo physical inactivity in the near future; (b) taking a baseline biological sample from the subject identified in (a) before or on the day the subject becomes physically inactive; (c) administering the intervention to the subject; (d) taking a test biological sample from the subject identified in (a) 3 or more days after the subject becomes physically inactive; (e) measuring the levels of at least one biomarker selected from MMP-1, MMP-3, and MMP-10 in the biological samples obtained by the sub-systems of (b) and (d); (f) calculating the difference in the levels of the at least one biomarker in the biological samples obtained by the sub-systems of (b) and (d); (g) comparing the one or more differences calculated by sub-system (f) to a control value based on the difference in levels of the at least one biomarker in comparable biological samples taken from one or more untreated control subjects at the beginning of and following a comparable period of physical inactivity; and (h) characterizing the intervention as efficacious if at least one of the following are true: (i) the difference for MMP-1 calculated by sub-system (f) is greater than the control value for MMP-1; (ii) the difference for MMP-3 calculated by sub-system (f) is smaller than the control value for MMP-3; and (iii) the difference for MMP-10 calculated by sub-system (f) is smaller than the control value for MMP-10.

In one exemplary embodiment, a composition comprising β-hydroxy-β-methylbutyric acid (HMB) or a salt, ester, or lactone thereof for use in treating or preventing the development of fibrosis in a subject who is undergoing or is expected to undergo a prolonged period of physical inactivity in the near future is provided. Use of the composition comprising HMB or a salt, ester, or lactone thereof may elevate the increase in circulating levels of MMP-1 that occurs with prolonged physical inactivity in untreated control subjects, and may attenuate the decrease in circulating levels of MMP-3 and MMP-10 that occur with prolonged physical inactivity in untreated control subjects. In one exemplary embodiment, the composition comprising HMB or a salt, ester, or lactone is a nutritional composition comprising at least one of: a source of protein, a source of carbohydrate, and a source of fat.

In one exemplary embodiment, a method of treating a subject at risk of developing fibrosis during a prolonged period of physical inactivity is provided. The method includes: (a) determining whether at least one of the following is true: (i) the MMP-1 level in a test biological sample of the subject taken during the prolonged period of physical inactivity is within 20% of the MMP-1 level in a baseline biological sample of the subject taken before or on the day the subject becomes physically inactive; (ii) the MMP-3 level in a test biological sample of the subject taken during the prolonged period of physical inactivity is lower than the MMP-3 level in a baseline biological sample of the subject taken before or on the day the subject becomes physically inactive; and (iii) the MMP-10 level in a test biological sample of the subject taken during the prolonged period of physical inactivity is lower than the MMP-10 level in a baseline biological sample of the subject taken before or on the day the subject becomes physically inactive; and (b) administering to the subject a therapeutically effective amount of HMB when at least one of (i)-(iii) is true. In one exemplary embodiment, the therapeutically effective amount of the HMB is administered to the subject via a nutritional composition comprising at least one of: a source of protein, a source of carbohydrate, and a source of fat.

### DETAILED DESCRIPTION

While the general inventive concepts are susceptible of embodiment in many different forms, described herein in detail are specific embodiments thereof with the understanding that the present disclosure is to be considered as an exemplification of the principles of the general inventive concepts. Accordingly, the general inventive concepts are not intended to be limited to the specific embodiments illustrated and described herein.

The terminology as set forth herein is for description of the exemplary embodiments only and should not be construed as limiting the disclosure as a whole. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise. Additionally, recitations of numerical ranges by endpoints include all numbers subsumed within that range (e.g., 1 to 5 includes 1, 1.5, 2, 2.75, 3, 3.80, 4, 5).

All publications, patent applications, patents, and other references mentioned herein are incorporated by reference in their entirety.

The exemplary methods and systems described herein are based, at least in part, on the inventors' discovery that levels of 3 circulating fibrosis biomarkers changed, i.e., increased or decreased, by statistically significant amounts in 18 healthy elderly subjects undergoing 10 days of bed rest. The exemplary methods are also based, at least in part, on the inventors' discovery that intervention with HMB altered the change in levels of 3 circulating biomarkers associated with fibrosis.

### Therapeutic Methods

In one exemplary embodiment, a method of attenuating the effect of prolonged physical inactivity on the development of fibrosis in a subject is provided. The exemplary method comprises administering a therapeutically effective amount of HMB to a subject who is experiencing a prolonged period of physical inactivity or who is expected to experience a prolonged period of physical inactivity in the near future, i.e., within the next few days, the next few weeks, or the next few months. For example, in one exemplary embodiment, the subject may be scheduled to undergo a procedure (e.g., surgery) that may lead to a prolonged period of physical inactivity within the next day, week, month, 2 days, 2 weeks, 2 months, 3 days, 3 weeks, 3 months, 4 days, 4 weeks, 4 months, 5 days, 5 weeks, 5 months, 6 days, 6 weeks, 6 months, or longer. As used herein, the term a “prolonged period of physical inactivity” refers to a period of physical inactivity that lasts 3 days or more. As used herein, the term “physical inactivity” refers to a condition or situation in which the subject seldom moves his or her limbs or body.

The exemplary methods and systems described herein may be used on a wide variety of subjects. For example, in one exemplary embodiment, the methods and systems may be used on a subject experiencing hospitalization. In one exemplary embodiment, the methods and systems may be used on a subject whose activities are restricted by others, such as a subject who is restricted to bed rest by a physician or other health care provider. In one exemplary embodiment, the methods and systems may be used on a subject whose activities are limited due to surgery, injury, infirmity, frailty, old age, and so forth. In one exemplary embodiment, the methods and systems may be used on a subject whose physical inactivity is self-imposed, such as a subject who is depressed.

The term “therapeutically effective amount,” as used herein, unless otherwise specified, refers to the amount of HMB which provides any therapeutic benefit in the prevention, treatment, or management of at least one of the symptoms, complications, or conditions associated with fibrosis or the development thereof. In one exemplary embodiment, HMB is first administered to the subject before the subject becomes physically inactive. In one exemplary embodiment, HMB is first administered when the subject becomes physically inactive, for example, on the day the subject becomes physically inactive or within a day or two after the subject becomes physically inactive. In one exemplary embodiment, HMB is administered to the subject throughout the entire period of physical inactivity. In one exemplary embodiment, HMB is administered to the subject before and throughout part or all of the period of physical inactivity. In one exemplary embodiment, HMB is administered to the subject after the period of physical inactivity has ended. In one exemplary embodiment, HMB is administered to the subject for up to one year. In one exemplary embodiment, HMB is administered to the subject for more than one year.

In one exemplary embodiment, the subject is a human subject. In one exemplary embodiment, the subject is an adult human subject. The phrase “adult human subject,” as used herein, unless otherwise specified, refers to a human that is at least 18 years of age or older. In one exemplary embodiment, the subject is an elderly human subject. The phrase “elderly human subject,” as used herein, unless otherwise specified, refers to a human that is at least 45 years of age, including at least 50 years of age, at least 55 years of age, at least 60 years of age, at least 65 years of age, at least 70 years of age, at least 75 years of age, and including at least 80 years of age or greater. The phrase “elderly human subject” also includes humans that are 45 years of age to 100 years of age, and humans that are 55 years of age to 80 years of age.

In one exemplary embodiment, the HMB is administered in an amount, in a manner, and for a time sufficient to alter the change in circulating levels of MMP-1, MMP-3, and MMP-10 that occurs with prolonged physical inactivity in untreated control subjects. In one exemplary embodiment, HMB is administered in an amount, in a manner, and for a time sufficient to enhance the increase in circulating levels of MMP-1 that occurs with prolonged physical inactivity in untreated control subjects. In one exemplary embodiment, HMB is administered in an amount, in a manner, and for a time sufficient to attenuate the decrease in circulating levels of MMP-3 that occurs with prolonged physical inactivity in untreated control subjects. In one exemplary embodiment, HMB is administered in an amount, in a manner, and for a time sufficient to attenuate the decrease in circulating levels of MMP-10 that occurs with prolonged physical inactivity in untreated control subjects. In one exemplary embodiment, HMB is administered in an amount, in a manner, and for a time sufficient to elevate the increase in circulating levels of MMP-1 that occurs with prolonged physical inactivity in untreated control subjects, to attenuate the decrease in circulating levels of MMP-3 that occurs with prolonged physical inactivity in untreated control subjects, to attenuate the decrease in circulating levels of MMP-10 that occurs with prolonged physical inactivity in untreated control subjects, or combinations thereof.

In one exemplary embodiment, HMB is administered in an amount, in a manner and for a time sufficient to keep a test level of at least one of MMP-3 and MMP-10 in a test biological sample taken from the subject following three or more days of physical inactivity comparable to a baseline level of at least one of MMP-3 and MMP-10 in a baseline biological sample taken from the subject at the beginning of or prior to the prolonged physical inactivity. As used herein, the test and baseline levels of the biomarkers in the test and baseline biological samples are “comparable” if they differ by 35% or less, including 25% or less, including 20% or less, including 15% or less, including 10% or less, including 5% or less, or 1% or less.

In one exemplary embodiment, a method of treating a subject at risk of developing fibrosis during a prolonged period of physical inactivity is provided. The exemplary method comprises: (a) determining whether at least one of the following is true: (i) the MMP-1 level in a test biological sample of the subject taken during the prolonged period of physical inactivity is within 20% of the MMP-1 level in a baseline biological sample of the subject taken before or on the day the subject becomes physically inactive; (ii) the MMP-3 level in a test biological sample of the subject taken during the prolonged period of physical inactivity is lower than the MMP-3 level in a baseline biological sample of the subject taken before or on the day the subject becomes physically inactive; and (iii) the MMP-10 level in a test biological sample of the subject taken during the prolonged period of physical inactivity is lower than the MMP-10 level in a baseline biological sample of the subject taken before or on the day the subject becomes physically inactive; and (b) administering to the subject a therapeutically effective amount of HMB when at least one of (i)-(iii) is true.

In one exemplary embodiment, the therapeutically effective amount of HMB is administered to the subject via a nutritional composition comprising at least one of: a source of protein, a source of carbohydrate, and a source of fat. In one exemplary embodiment, the therapeutically effective amount of HMB is co-administered with alpha-ketoisocaproate (KIC), alpha-hydroxyisocaproate (HICA), or both. In one exemplary embodiment, the therapeutically effective amount of HMB is co-administered with KIC, HICA, or both via a nutritional composition comprising at least one of: a source of protein, a source of carbohydrate, and a source of fat.

### HMB

The term HMB, which is also referred to as β-hydroxy-β-methylbutyric acid, or β-hydroxy-isovaleric acid, can be represented in its free acid form as (CH₃)₂(OH)CCH₂COOH. HMB is a metabolite of leucine formed by transamination to a-ketoisocaproate (KIC) in muscle followed by oxidation of the KIC in the cytosol of the liver to give HMB. A variety of suitable forms of HMB may be used in the exemplary methods and systems described herein. For example, in one exemplary embodiment, HMB is selected from the group consisting of a free acid, a salt, an ester, and a lactone. In one exemplary embodiment, HMB is in the form of a non-toxic, edible salt. In one exemplary embodiment, the HMB salt is water-soluble or becomes water-soluble in the stomach or intestines of a subject. In one exemplary embodiment, the HMB salt is selected from a sodium salt, a potassium salt, a magnesium salt, a chromium salt, and a calcium salt. However, in certain other embodiments, other non-toxic salts, such as other alkali metal or alkaline earth metal salts of HMB, may be used.

In one exemplary embodiment, a pharmaceutically acceptable ester of HMB may be used in the methods and systems described herein. The HMB ester may be rapidly converted to HMB in its free acid form upon consumption by a subject. In one exemplary embodiment, the HMB ester is a methyl ester or an ethyl ester. HMB methyl ester and HMB ethyl ester are typically rapidly converted to the free acid form of HMB upon consumption by a subject. In one exemplary embodiment, a pharmaceutically acceptable lactone may be used in the methods and systems described herein. The HMB lactone may be rapidly converted to HMB in its free acid form upon consumption by a subject. In one exemplary embodiment, the HMB lactone is an isovaleryl lactone or a similar lactone, which typically are rapidly converted to the free acid form of HMB upon consumption by a subject.

Methods for producing HMB and its derivatives are well known in the art. For example, HMB can be synthesized by oxidation of diacetone alcohol. One suitable procedure is described by Coffman et al., J. Am. Chem. Soc. 80: 2882-2887 (1958). As described therein, HMB is synthesized by an alkaline sodium hypochlorite oxidation of diacetone alcohol. The product is recovered in free acid form, which can be converted to the desired salt. For example, 3-hydroxy-3-methylbutyric acid (HMBA) can be synthesized from diacetone alcohol (4-hydroxy-4-methylpentan-2-one) via oxidation using cold, aqueous hypochlorite (bleach). After acidifying the reaction mixture using HCl, the HMBA product is recovered by extraction using ethyl acetate, and separating and retaining the organic layer from the extraction mixture. The ethyl acetate is removed by evaporation and the residue dissolved in ethanol. After addition of Ca(OH)₂ and cooling, crystalline Ca-HMB can be recovered by filtration, the crystals washed with ethanol and then dried. Alternatively, the HMB can be obtained from a commercial source. For example, the calcium salt of HMB is commercially available from Technical Sourcing International (TSI) of Salt Lake City, Utah.

The routes for administering HMB include an oral diet, tube feeding, and peripheral or total parenteral nutrition. In one exemplary embodiment, the HMB or source thereof is administered to the subject orally. In certain embodiments, the HMB or source thereof is administered to the subject via tube feeding by means of nasogastric, nasoduodenal, esophagostomy, gastrostomy, or jejunostomy tubes.

In one exemplary embodiment, the HMB may be administered alone, without a carrier. For example, the HMB may be dissolved in water and consumed by the subject. Alternatively, the HMB may be sprinkled on food, dissolved in coffee, and so forth. The total daily dose for the subject will vary widely, but typically a subject will benefit from consuming at least 2 g/day of HMB, including 3 g/day to 10 g/day of HMB, or 4 g/day to 8 g/day of HMB. Alternatively, in one exemplary embodiment, the total daily dose of HMB may be 20 mg/kg of body weight/day to 40 mg/kg of body weight/day.

In one exemplary embodiment, the HMB may be incorporated into pills, capsules, rapidly dissolved tablets, lozenges, and so forth. The active dose can vary widely, but will typically be 250 mg/dose to 1 g/dose with the subject consuming between 2 and 8 doses per day to achieve a target of 2 g/day minimum. Methods for preparing such dosage forms are well known in the art. The reader's attention is directed to the most recent edition of Remington's Pharmaceutical Sciences for guidance on how to prepare such dosage forms.

In one exemplary embodiment, the HMB may be combined with additional supplements such as amino acids. One example of such a supplement is Juven® (Abbott Nutrition, Columbus, Ohio), a powder (sachet) containing 1.5 grams of Ca-HMB, 7 grams of arginine, and 7 grams of glutamine.

### Nutritional Matrices

Although the HMB may be administered as a single entity without a carrier, the HMB may also be incorporated into food products and consumed by the subject during their meals or snacks. In one exemplary embodiment, the HMB may be administered to the subject via a nutritional composition. In other words, the HMB may be incorporated into a nutritional composition, which may then be administered to the subject.

The term “nutritional composition,” as used herein, unless otherwise specified, refers to nutritional products in various forms including, but not limited to, liquids, solids, powders, semi-solids, semi-liquids, nutritional supplements, meal replacement products, and any other nutritional food product known in the art. A nutritional composition in powder form may often be reconstituted to form a nutritional composition in liquid form.

Generally, the nutritional composition includes one or more ingredients that help satisfy the subject's nutritional requirements, in addition to providing a useful vehicle for the delivery of HMB. For example, the nutritional composition can include protein, carbohydrate, fat, and combinations thereof. In certain embodiments, the nutritional composition includes at least one source of protein, at least one source of carbohydrate, and at least one source of fat. Many different sources and types of protein, carbohydrate, and fat are known and can be used in the nutritional composition. In certain embodiments, the nutritional composition is in the form of a ready-to-drink liquid, a powder suitable for reconstitution to a liquid, or a bar. The nutritional composition is generally suitable for oral consumption by a human.

In one exemplary embodiment, the HMB may be incorporated into a meal replacement beverage. In one exemplary embodiment, the HMB may be incorporated into a meal replacement bar. In one exemplary embodiment, the HMB may be incorporated into a juice, a carbonated beverage, bottled water, and so forth. In one exemplary embodiment, the HMB may be incorporated into a medical nutritional product designed to support specific disease states. Methods for producing any such nutritional compositions are well known to those skilled in the art. The following discussion is intended to illustrate such exemplary nutritional compositions and their preparation.

Most meal replacement products (e.g., bars or liquids) provide calories from protein, carbohydrates, and fat. Typically, such meal replacement products also contain vitamins and minerals, because they are intended to be suitable for use as a sole source of nutrition. While such meal replacement products may serve as a sole source of nutrition, they often are not used in this manner. Rather, individuals consume the meal replacement products in a supplemental fashion to replace one or two meals a day, or to provide a healthy snack. The nutritional compositions described herein should be construed to include any of these exemplary embodiments.

As previously discussed, the nutritional composition will typically contain suitable proteins, carbohydrates, and fats as is known to those skilled in the art of making nutritional compositions. Proteins suitable for use in the nutritional composition include, but are not limited to, hydrolyzed, partially hydrolyzed, or intact proteins or protein sources, and can be derived from any known or otherwise suitable source such as milk (e.g., casein, whey), animal (e.g., meat, fish), cereal (e.g., rice, corn), vegetable (e.g., soy, potato, pea), egg (egg albumin), gelatin, or combinations thereof. Suitable intact protein sources include, but are not limited to, soy based, milk based, casein protein, whey protein, rice protein, beef collagen, earthworm protein, insect protein, potato protein, pea protein, or combinations thereof.

Optionally, the intact protein source is enriched in large neutral amino acids (LNAA) comprising valine, isoleucine, leucine, threonine, tyrosine and phenylalanine. Typically, about 40% of casein, whey, and soy protein sources are large neutral amino acids. For example, caseinate contains about 38 wt/wt % LNAA, whey protein concentrate contains about 39 wt/wt % LNAA, and soy protein isolate contains about 34 wt/wt % LNAA. In certain embodiments, the nutritional composition is formulated with a protein source that will deliver about 1 gram to 25 grams of LNAA per day, about 1 gram to 20 grams of LNAA per day, or about 4 grams to 20 grams of LNAA per day. As an example, a nutritional composition consumed 3 times a day that contains a protein comprising 4.8 grams of LNAA will deliver 14.4 grams of LNAA per day.

Suitable carbohydrates for use in the nutritional composition include, but are not limited to, hydrolyzed, intact, naturally or chemically modified starches sourced from corn, tapioca, rice, or potato in waxy or non waxy forms; and sugars such as glucose, fructose, lactose, sucrose, maltose, high fructose corn syrup, corn syrup solids, fructooligosaccharides, and combinations thereof.

Suitable fats for use in the nutritional compositions include, but are not limited to, canola oil, corn oil, coconut oil, fractionated coconut oil, soy oil, olive oil, safflower oil, high gamma-linolenic acid (GLA) safflower oil, high oleic safflower oil, medium chain triglycerides (MCT) oil, sunflower oil, high oleic sunflower oil, palm and palm kernel oils, palm olein, marine oils, cottonseed oils, algal and fungal derived oils, and combinations thereof.

The nutritional composition will typically contain vitamins and minerals in an amount designed to supply or supplement the daily nutritional requirements of the subject consuming the nutritional composition. Those skilled in the art will recognize that nutritional products often include overages of certain vitamins and minerals to ensure that they meet a targeted level over the shelf life of the product. These same individuals will also recognize that certain micro ingredients may have potential benefits for people depending upon any underlying illness or disease that the subject is afflicted with. For example, cancer patients benefit from antioxidants such as beta-carotene, Vitamin C, Vitamin E, and selenium. In certain embodiments, the nutritional composition comprises the following vitamins and minerals: calcium; phosphorus; sodium; chloride; magnesium; manganese; iron; copper; zinc; selenium; iodine; chromium; molybdenum; conditionally essential nutrients m-inositol, carnitine, and taurine; Vitamins A, C, D, E, K and the B complex; and mixtures thereof.

In certain embodiments, the nutritional composition may also contain oligosaccharides such as fructooligosaccharides (FOS) or galactooligosaccharides (GOS). Oligosaccharides are rapidly and extensively fermented to short chain fatty acids by anaerobic microorganisms that inhabit the large bowel. These oligosaccharides are preferential energy sources for most Bifidobacterium species, but are not utilized by potentially pathogenic organisms such as Clostridium perfringens, C. difficile, or Escherichia coli.

Typically, the FOS comprises 0 grams/serving to 5 grams/serving of the nutritional composition, including 1 gram/serving to 5 grams/serving, or 2 grams/serving to 4 grams/serving of the nutritional composition.

The nutritional composition may also contain a flavor to enhance its palatability. Artificial sweeteners may be added to complement the flavor and mask undesirable (e.g., salty) taste. Useful artificial sweeteners include saccharine, stevia, sucralose, acesulfame-K (ace-K), and so forth.

In one exemplary embodiment, the nutritional composition is a solid. Solid nutritional compositions such as bars, cookies, and so forth may also be manufactured utilizing techniques known to those skilled in the art. For example, solid nutritional compositions may be manufactured using cold extrusion technology as is known in the art. To prepare such a solid composition, typically all of the powdered components are dry blended together. Such powdered components typically include the proteins, vitamin premixes, certain carbohydrates, and so forth. The fat-soluble components are then blended together and mixed with the powdered premix above. Finally any liquid components are then mixed into the composition, forming a plastic like composition or dough.

The process above is intended to give a plastic mass that can then be shaped, without further physical or chemical changes occurring, by the procedure known as cold forming or extrusion. In this process, the plastic mass is forced at relatively low pressure through a die, which confers the desired shape. The resultant extrudate is then cut off at an appropriate position to give products of the desired weight. If desired, the solid nutritional composition may be coated, to enhance palatability, and packaged for distribution.

The solid nutritional composition may also be manufactured through a baked application or heated extrusion to produce cereals, cookies, and crackers. One knowledgeable in the arts would be able to select from among many suitable manufacturing processes.

As previously discussed, the HMB may be incorporated into beverages such as juices, non-carbonated beverages, carbonated beverages, electrolyte solutions, flavored waters, and so forth. The HMB will typically comprise 0.5 grams/serving to 2 grams/serving of the beverages. Methods for producing such beverages are well known in the art. The reader's attention is directed to U.S. Pat. Nos. 6,176,980 and 5,792,502, the entire contents of each being hereby incorporated by reference. For example, all of the ingredients, including the HMB could be dissolved in an appropriate volume of water. Then, any flavors, colors, vitamins, and so forth are added. The mixture is subsequently pasteurized, packaged, and stored until shipment.

In certain embodiments, the nutritional composition is formulated as a clear liquid having a pH between 2 and 5, and also having no more than 0.5% fat by weight of the nutritional composition. The limited amount of fat contributes to the desired clarity of the nutritional composition. Typically, a liquid nutritional composition that is formulated to be clear, or at least substantially translucent, is substantially free of fat. As used herein “substantially free of fat” refers to a nutritional composition that contains less than 0.5% fat by weight of the composition, or less than 0.1% fat by weight of the composition. “Substantially free of fat” also may refer to a nutritional composition that contains no fat, i.e., zero fat. A liquid nutritional composition that is both clear and has a pH between 2 and 5 is also typically substantially free of fat. In certain embodiments, the pH of the nutritional composition may be between 2.5 and 4.6, including a pH between 3 and 3.5. In certain embodiments of the nutritional compositions that are substantially free of fat but have some amount of fat present, the fat may be present as a result of being inherently present in another ingredient (e.g., a source of protein) or may be present as a result of being added as one or more separate sources of fat.

### Dosing

The amount of HMB that is sufficient to reduce fibrosis or the development thereof associated with prolonged physical inactivity in a human subject can be determined in clinical studies that employ a population of control subjects. The dosing can also be optimized to the subject undergoing a prolonged period of physical inactivity. In one exemplary embodiment, the dosing is optimized to the subject undergoing a prolonged period of physical inactivity by monitoring the circulating levels of the MMP-1, MMP-3, and MMP-10 biomarkers over the course of the period of physical inactivity and evaluating the efficacy of the HMB intervention as described below.

In certain embodiments, when the HMB or a nutritional composition comprising HMB is orally administered about twice a day for a minimum of two weeks, the dose is sufficient to provide at least about 2 grams per day of HMB, for example, between 1 gram and 10 grams per day for a typical 70 kilogram person, or between about 2 grams and 5 grams per day of HMB. The dosing on a body weight basis may be between about 0.01 grams and about 0.10 grams per kilogram body weight, or between about 0.02 grams and 0.07 grams per kilogram body weight.

### Evaluation Methods

Exemplary methods and systems for evaluating the efficacy of an intervention on the development of fibrosis in a subject who is undergoing or is expected to undergo a prolonged period of physical inactivity in the near future are also provided herein. In one exemplary embodiment, the method for evaluating the efficacy of an intervention on the development of fibrosis in a subject who is undergoing or is expected to undergo a prolonged period of physical inactivity in the near future comprises: (a) measuring levels of at least one biomarker selected from MMP-1, MMP-3, and MMP-10 in a baseline biological sample taken from the subject before or on the day the subject becomes physically inactive; (b) administering the intervention to the subject, wherein the intervention is first administered to the subject before or on the day the subject becomes physically inactive; (c) measuring levels of the at least one biomarker measured in (a) in a test biological sample taken from the subject at three or more days after the subject has become physically inactive; (d) calculating the difference between the levels of the at least one biomarker measured in the baseline biological sample and the at least one biomarker measured in the test biological sample; (e) comparing the differences calculated in (d) to corresponding control values based on the difference between levels of the at least one biomarker in comparable biological samples taken from untreated control subjects at comparable time points; and (f) determining that the intervention is efficacious when: (i) the difference for MMP-1 calculated in (d) is greater than the control value for MMP-1; (ii) the difference for MMP-3 calculated in (d) is smaller than the control value for MMP-3; (iii) the difference for MMP-10 calculated in (d) is smaller than the control value for MMP-10; or (iii) combinations of (i), (ii), and (iii).

In one exemplary embodiment, the evaluation method utilizes MMP-1 as one of the one or more biomarkers. As shown in Table 1 below, circulating levels of MMP-1 increase in untreated control subjects who have experienced ten days of bed rest. Interventions that elevate the increase in MMP-1 levels by a statistically significant amount are efficacious.

In one exemplary embodiment, the evaluation method utilizes MMP-3 as one of the one or more biomarkers. As shown in Table 1 below, circulating levels of MMP-3 decrease in untreated control subjects who have experienced ten days of bed rest. Interventions that attenuate or otherwise mitigate the decrease in MMP-3 levels by a statistically significant amount are efficacious.

In one exemplary embodiment, the evaluation method utilizes MMP-10 as one of the one or more biomarkers. As shown in Table 1 below, circulating levels of MMP-10 decrease in untreated control subjects who have experienced ten days of bed rest. Interventions that attenuate or otherwise mitigate the decrease in MMP-10 levels by a statistically significant amount are efficacious.

In one exemplary embodiment, the intervention is first administered to the subject at the beginning of the period of physical inactivity, for example, on the day the subject becomes physically inactive. In one exemplary embodiment, the intervention is first administered to the subject before the subject becomes physically inactive, for example, one day, two days, one week, or two weeks before the subject becomes physically inactive. In one exemplary embodiment, the intervention is administered to the subject continuously throughout the entire period of physical inactivity. In certain embodiments, the intervention is administered to the subject throughout the entire period of physical inactivity and after the period of physical inactivity ends. In one exemplary embodiment, the intervention is administered to the subject before the period of physical inactivity begins and throughout the entire period of physical inactivity. Thus, the period of time that the intervention is administered to the subject may be longer than the period of physical inactivity.

The baseline biological sample is used to determine baseline values for the at least one biomarker in a subject. Thus, in one exemplary embodiment, the baseline biological sample may be taken from the subject before or on the day the subject becomes physically inactive. In one exemplary embodiment, a test biological sample is taken from the subject at three or more days after the subject becomes physically inactive (and three or more days after the intervention has commenced). In one exemplary embodiment, a test biological sample is taken from the subject ten or more days after the subject becomes physically inactive (and ten or more days after the intervention has commenced). In one exemplary embodiment, a test biological sample is taken from the subject three to seven days after the subject becomes physically inactive (and three to seven days after the intervention has commenced). In one exemplary embodiment, a test biological sample is taken from the subject one to four weeks after the subject becomes physically inactive (and one to four weeks after the intervention has commenced). In one exemplary embodiment, a test biological sample is taken from the subject one to three months after the subject becomes physically inactive (and one to three months after the intervention has commenced). In one exemplary embodiment, multiple (e.g., two, three, four) test biological samples are taken from the subject at various time periods after the subject becomes physically inactive and after the intervention has commenced. For example, a test biological sample may be taken from the subject at one week, two weeks, and three weeks after the subject has become physically inactive.

In one exemplary embodiment, a method for evaluating the efficacy of an intervention on the development of fibrosis in a subject who is undergoing or is expected to undergo a prolonged period of physical inactivity in the near future comprises: (a) measuring levels of at least one biomarker selected from MMP-1, MMP-3, and MMP-10 in a baseline biological sample taken from the subject before the subject becomes physically inactive; (b) administering the intervention to the subject, wherein the intervention is first administered to the subject after the subject becomes physically inactive; (c) measuring levels of the at least one biomarker measured in (a) in a test biological sample taken from the subject after the intervention has been administered to the subject; (d) calculating the difference between the levels of the at least one biomarker measured in the baseline biological sample and the at least one biomarker measured in the test biological sample; and (e) determining that the intervention is efficacious when the measured level of the at least one biomarker in the test biological sample is comparable to the measured level of the at least one biomarker in the baseline biological sample. As described herein, the level of the at least one biomarker is comparable if the difference between the baseline level of the biomarker and the level measured in the test biological sample is 35% or less, including 25% or less, including 20% or less, including 15% or less, including 10% or less, including 5% or less, or 1% or less.

In one exemplary embodiment, a method for evaluating the efficacy of an intervention on the development of fibrosis in a subject who is undergoing or is expected to undergo a prolonged period of physical inactivity in the near future comprises: (a) administering the intervention to the subject, wherein the intervention is first administered to the subject before or on the day the subject becomes physically inactive; (b) measuring the level of at least one biomarker selected from MMP-1, MMP-3, and MMP-10 in a test biological sample taken from the subject three or more days after the subject becomes physically inactive; (c) comparing the levels measured in (b) to a baseline level of the at least one biomarker in a baseline biological sample taken from the subject before or on the day the subject becomes physically inactive; and (d) determining that the intervention is efficacious when the measured level of the at least one biomarker in the test biological sample is comparable to the baseline level of the at least one biomarker in the baseline biological sample. As described herein, the level of the at least one biomarker is comparable if the difference between the baseline level of the biomarker and the level measured in the test biological sample is 35% or less, including 25% or less, including 20% or less, including 15% or less, including 10% or less, including 5% or less, or 1% or less.

### Biological Samples

Biological samples suitable for use in the exemplary methods include, but are not limited, to blood samples, including whole blood samples, and samples of blood fractions, including but not limited to, serum and plasma. The sample may be fresh blood or stored blood (e.g., from a blood bank) or blood fractions. The biological sample may be a blood sample expressly obtained for the assays associated with the methods described herein, or a blood sample obtained for another purpose that can be sub-sampled for the assays associated with the methods described herein.

In one exemplary embodiment, the blood sample is whole blood. Whole blood may be obtained from the subject using standard clinical procedures. In one exemplary embodiment, the blood sample is plasma. Plasma may be obtained from whole blood samples by centrifugation of anti-coagulated blood. Such a process provides a buffy coat of white cell components and a supernatant of the plasma. In one exemplary embodiment, the blood sample is serum. Serum may be obtained by centrifugation of whole blood samples that have been collected in tubes that are free of anti-coagulant. The blood is permitted to clot prior to centrifugation. The yellowish-reddish fluid that is obtained by centrifugation is the serum.

The blood sample may be pretreated as necessary by dilution in an appropriate buffer solution, heparinized, concentrated if desired, or fractionated by any number of methods including, but not limited to, ultracentrifugation or fractionation by fast performance liquid chromatography (FPLC). A wide variety of standard aqueous buffer solutions, employing one of a number of buffers, such as phosphate, Tris, or the like, at physiological pH can be used.

Other biological samples that may be used in the exemplary methods include, but are not limited to, urine and saliva.

### Measuring Levels of the Biomarkers

Levels of each of the biomarkers in the subject's biological samples can be determined by various methods such as by using polyclonal or monoclonal antibodies that are immunoreactive with the respective biomarker or by using other binding agents such as aptamers or protein domains suitable for binding target from phase display libraries. For example, antibodies immunospecific for MMP-3 may be made and labeled using standard procedures and then employed in immunoassays to detect the presence of MMP-3 in a biological sample. Suitable immunoassays include, by way of example, radioimmunoassays, both solid and liquid phase, fluorescence-linked assays, competitive immunoassays, or enzyme-linked immunosorbent assays. In certain embodiments, the immunoassays may also be used to quantify the amount of the biomarker that is present in the biological sample.

Each of the biomarkers can be used as an immunogen to produce antibodies immunospecific for the oxidized protein or peptide fragment. The term “immunospecific,” as used herein, means the antibodies have substantially greater affinity for the immunogen than for other proteins. Such antibodies may include, but are not limited to, polyclonal, monoclonal, chimeric, single chain, and Fab fragments.

Antibodies raised against the select biomarker species may be produced according to established procedures. Generally, the biomarker is used to immunize a host animal. Suitable host animals, include, but are not limited to, rabbits, mice, rats, goats, and guinea pigs. Various adjuvants may be used to increase the immunological response in the host animal. The adjuvant used depends, at least in part, on the host species. Such animals produce heterogenous populations of antibody molecules, which are referred to as polyclonal antibodies and which may be derived from the sera of the immunized animals.

Polyclonal antibodies may be generated using conventional techniques by administering the biomarker to a host animal. Depending on the host species, various adjuvants may be used to increase the immunological response. For example, Bacille Calmette-Guérin (BCG) and Corynebacterium parvum may be used as adjuvants in humans. Conventional protocols may also be used to collect blood from the immunized animals and to isolate the serum or the IgG fraction from the collected blood.

For preparation of monoclonal antibodies, conventional hybridoma techniques may be used. Such antibodies are produced by continuous cell lines in culture. Suitable techniques for preparing monoclonal antibodies include, but are not limited to, the hybridoma technique, the human B-cell hybridoma technique, and the EBV hybridoma technique.

Various immunoassays may be used for screening to identify antibodies having the desired specificity. These include protocols that involve competitive binding or immunoradiometric assays and typically involve the measurement of complex formation between the respective biomarker and the antibody.

The present antibodies may be used to detect the presence of or measure the amount of biomarker in a biological sample taken from the subject. In one exemplary embodiment, the method comprises contacting a biological sample taken from the subject with one or more of the present antibodies; and assaying for the formation of a complex between the antibody and the biomarker in the biological sample. For ease of detection, the antibody can be attached to a substrate such as a column, plastic dish, matrix, or membrane, such as nitrocellulose. In certain embodiments, the method employs an enzyme-linked immunosorbent assay (ELISA) or a Western immunoblot procedure.

The presence or amount of one or more biomarkers can be determined using antibodies that specifically bind to each marker as well as any additional biomarkers if such additional biomarkers are used. Examples of antibodies that can be used include a polyclonal antibody, a monoclonal antibody, a human antibody, an immunoglobulin molecule, a disulfide linked Fv, an affinity matured antibody, a scFv, a chimeric antibody, a single domain antibody, a CDR-grafted antibody, a diabody, a humanized antibody, a multispecific antibody, a Fab, a dual specific antibody, a DVD, a Fab′, a bispecific antibody, a F(ab′)2, a Fv, and combinations thereof. For example, the immunological method may include measuring the levels of a biomarker by: (i) contacting a test sample with at least one capture antibody, wherein the capture antibody binds to an epitope on the biomarker or a fragment thereof to form a capture antibody-antigen complex; (ii) contacting the capture antibody-antigen complex with at least one detection antibody comprising a detectable label, wherein the detection antibody binds to an epitope on the biomarker (antigen) that is not bound by the capture antibody and forms a capture antibody-antigen-detection antibody complex; and (iii) determining the biomarker level in the test sample based on the signal generated by the detectable label in the capture antibody-antigen-detection antibody complex formed in (ii). A wide variety of immunoassay techniques may be utilized. For example, the immunoassay may be an enzyme-linked immunoassay (ELISA); a radioimmunoassay (RIA); a competitive inhibition assay, such as forward or reverse competitive inhibition assays; a fluorescence polarization assay; or a competitive binding assay. The ELISA may be a sandwich ELISA. Specific immunological binding of the antibody to the marker can be detected via direct labels, such as fluorescent or luminescent tags, metals and radionuclides attached to the antibody or via indirect labels, such as alkaline phosphatase or horseradish peroxidase.

The use of immobilized antibodies or fragments thereof may be incorporated into the immunoassay. The antibodies may be immobilized onto a variety of supports, such as magnetic or chromatographic matrix particles, the surface of an assay plate (such as microtiter wells), pieces of a solid substrate material, and the like. An assay strip can be prepared by coating the antibody or a plurality of antibodies in an array on a solid support. This strip can then be dipped into the biological sample and then processed quickly through washes and detection steps to generate a measurable signal, such as a colored spot.

The sandwich ELISA measures the amount of antigen between two layers of antibodies (i.e., a capture antibody and a detection antibody (which may be labeled with a detectable label)). The marker to be measured may contain at least two antigenic sites capable of binding to antibody. Either monoclonal or polyclonal antibodies may be used as the capture and detection antibodies in the sandwich ELISA.

Generally, at least two antibodies are employed to separate and quantify the biomarker of interest (as well as any additional biomarkers), in a biological sample. More specifically, the at least two antibodies bind to certain epitopes of the biomarker forming an immune complex which is referred to as a “sandwich.” One or more antibodies can be used to capture the biomarker in the biological sample (these antibodies are frequently referred to as a “capture” antibody or “capture” antibodies) and one or more antibodies can be used to bind a detectable (namely, quantifiable) label to the sandwich (these antibodies are frequently referred to as a “detection” antibody or “detection” antibodies). In a sandwich assay, both antibodies binding to their epitope may not be diminished by the binding of any other antibody in the assay to its respective epitope. In other words, antibodies may be selected so that the one or more first antibodies brought into contact with a biological sample suspected of containing the marker do not bind to all or part of an epitope recognized by the second or subsequent antibodies, thereby interfering with the ability of the one or more second detection antibodies to bind to the marker.

In one exemplary embodiment, a biological sample suspected of containing the marker can be contacted with at least one first capture antibody (or antibodies) and at least one second detection antibody, either simultaneously or sequentially. In the sandwich assay format, a biological sample suspected of containing the marker is first brought into contact with the at least one first capture antibody that specifically binds to a particular epitope under conditions which allow the formation of a first antibody-marker complex. If more than one capture antibody is used, a first multiple capture antibody-marker complex is formed. In a sandwich assay, the antibodies, such as the at least one capture antibody, are used in molar excess amounts of the maximum amount of marker expected in the biological sample.

In certain embodiments, prior to contacting the biological sample with the at least one first capture antibody, the at least one first capture antibody can be bound to a solid support which facilitates the separation of the first antibody-marker complex from the biological sample. Any solid support known in the art can be used, including but not limited to, solid supports made out of polymeric materials in the form of wells, tubes, or beads. The antibody (or antibodies) can be bound to the solid support by adsorption, by covalent bonding using a chemical coupling agent, or by other means known in the art, provided that such binding does not interfere with the ability of the antibody to bind the marker. Moreover, if necessary, the solid support can be derivatized to allow reactivity with various functional groups on the antibody. Such derivatization requires the use of certain coupling agents such as, but not limited to, maleic anhydride, N-hydroxysuccinimide, and 1-ethyl-3-(3-dimethylaminopropyl)carbodiimide.

After the biological sample suspected of containing the marker is brought into contact with the at least one first capture antibody, the biological sample is incubated in order to allow for the formation of a first capture antibody (or multiple antibody)-marker complex. The incubation can be carried out at a pH between about 4.5 and about 10.0, at a temperature between about 2° C. and about 45° C., and for a period between at least about one (1) minute and about eighteen (18) hours, including about two minutes to about six minutes, and including about three minutes to about four minutes.

After formation of the first/multiple capture antibody-marker complex, the complex is then contacted with at least one second detection antibody (under conditions which allow for the formation of a first/multiple antibody-marker second antibody complex). If the first antibody-marker complex is contacted with more than one detection antibody, then a first/multiple capture antibody-marker-multiple antibody detection complex is formed. As with the first antibody, when the at least second (and subsequent) antibody is brought into contact with the first antibody-marker complex, a period of incubation under conditions similar to those described above is required for the formation of the first/multiple antibody-marker-second/multiple antibody complex. In certain embodiments, at least one second antibody contains a detectable label. The detectable label can be bound to the at least one second antibody prior to, simultaneously with, or after the formation of the first/multiple antibody-marker-second/multiple antibody complex. Any detectable label known in the art can be used.

### Kits for Performing the Methods

In one exemplary embodiment, a kit may be used for performing the exemplary methods described above. The kit may comprise: (1) reagents capable of specifically binding to the biomarker to quantify the levels of the marker in a biological sample taken from a subject wherein at least one reagent comprises an antibody capable of specifically binding the marker; and (2) a reference standard indicating a reference level of the biomarker. The kit may further comprise at least one reagent (e.g., an antibody) capable of specifically binding at least one additional biomarker, and a reference standard indicating a reference level of the at least one additional biomarker of the condition being assessed, if present.

In one exemplary embodiment, the kit may comprise antibodies and a means for administering the antibodies. In one exemplary embodiment, the kit may further comprise instructions for using the kit and conducting the analysis, monitoring, or subsequent treatment.

In one exemplary embodiment, the kit may further comprise one or more containers, such as vials or bottles, with each container containing a separate reagent. In one exemplary embodiment, the kit may further comprise written instructions, which may describe how to perform or interpret an analysis, monitoring, treatment, or method described herein.

For example, the kit can comprise instructions for assaying the biological sample for one or more biomarkers by immunoassay, for example, chemiluminescent microparticle immunoassay. The instructions can be in paper form or machine-readable form, such as a disk, CD, DVD, or the like. The antibody can be a detection antibody (meaning an antibody labeled with a detectable label). For example, the kit can contain at least one capture antibody that specifically binds the antigen or biomarker of interest. The kit can also contain a conjugate antibody (such as an antibody labeled with a detectable label) for each capture antibody. Alternatively or additionally, the kit can comprise a calibrator or control, for example, purified, and optionally lyophilized, or a container (e.g., tube, microtiter plate, or strip, which is already coated with an anti-biomarker monoclonal antibody) for conducting the assay. Moreover, the kit can comprise a buffer, such as an assay buffer or a wash buffer, either one of which can be provided as a concentrated solution, a substrate solution for the detectable label (e.g., an enzymatic label), or a stop solution. In one exemplary embodiment, the kit comprises all components (e.g., reagents, standards, buffers, diluents) which are necessary to perform the assay. The instructions also can include instructions for generating a standard curve or a reference standard for purposes of quantifying the biomarker of interest.

As alluded to above, any antibodies, which are provided in the kit, such as recombinant antibodies specific for the biomarker, can incorporate a detectable label, such as a fluorophore, radioactive moiety, enzyme, biotin/avidin label, chromophore, chemiluminescent label, or the like, or the kit can include reagents for labeling the antibodies or reagents for detecting the antibodies (e.g., the detection antibodies) or for labeling the analytes or reagents for detecting the analyte. The antibodies, calibrators, or controls can be provided in separate containers or pre-dispensed into an appropriate assay format, for example, into microtiter plates.

Optionally, the kit may include quality control components (for example, sensitivity panels, calibrators, and positive controls). Preparation of quality control reagents is well-known in the art and is described on insert sheets for a variety of immunodiagnostic products. Sensitivity panel members optionally are used to establish assay performance characteristics, and further optionally are useful indicators of the integrity of the immunoassay kit reagents, and the standardization of assays.

The kit can also optionally include other reagents required to conduct a diagnostic assay or facilitate quality control evaluations, such as buffers, salts, enzymes, enzyme co-factors, substrates, detection reagents, and the like. Other components, such as buffers and solutions for the isolation or treatment of a biological sample (e.g., pretreatment reagents), may also be included in the kit. The kit may additionally include one or more other controls. One or more of the components of the kit may be lyophilized, in which case the kit may further comprise reagents suitable for the reconstitution of the lyophilized components.

The various components of the kit optionally are provided in suitable containers as necessary, for example, a microtiter plate. The kit can further include containers for holding or storing a sample (e.g., a container or cartridge for a blood sample). Where appropriate, the kit optionally may contain reaction vessels, mixing vessels, and other components that facilitate the preparation of reagents or the biological sample. The kit may also include one or more instruments for assisting with obtaining a test sample, such as a syringe, pipette, forceps, measured spoon, or the like.

If the detectable label is an acridinium compound, the kit can comprise at least one acridinium-9-carboxamide, at least one acridinium-9-carboxylate aryl ester, or any combination thereof. If the detectable label is an acridinium compound, the kit also can comprise a source of hydrogen peroxide, such as a buffer, solution, or at least one basic solution.

In certain embodiments, the kit may contain a solid phase, such as a magnetic particle, bead, test tube, microtiter plate, cuvette, membrane, scaffolding molecule, film, filter paper, a quartz crystal, disc, or chip. The kit may also include a detectable label that can be or is conjugated to an antibody, such as an antibody functioning as a detection antibody. The detectable label can be, for example, a direct label, which may be an enzyme, oligonucleotide, nanoparticle, chemiluminophore, fluorophore, fluorescence quencher, chemiluminescence quencher, or biotin. In certain embodiments, the kit may include any additional reagents needed for detecting the label.

In certain embodiments, the kit may further comprise one or more components, alone or in further combination with instructions, for assaying the biological sample for another analyte, which can be a biomarker, such as a biomarker of another condition of interest. A sample, such as a serum sample, can also be assayed for an additional biomarker using TOF-MS and an internal standard.

The kit (or components thereof), as well as the method of determining the concentration of the biomarker in a biological sample by an immunoassay as described herein, can be adapted for use in a variety of automated and semi-automated systems (including those where the solid phase comprises a microparticle), as described, for example, in U.S. Pat. Nos. 5,089,424 and 5,006,309, and as commercially marketed, for example, by Abbott Laboratories (Abbott Park, Ill.) as ARCHITECT®.

Some of the differences between an automated or semi-automated system as compared to a non-automated system (e.g., ELISA) include the substrate to which the first specific binding partner (e.g., analyte antibody or capture antibody) is attached (which can impact sandwich formation and analyte reactivity), and the length and timing of the capture, detection, and any optional wash steps. Whereas a non-automated format such as an ELISA may require a relatively longer incubation time with sample and capture reagent (e.g., about 2 hours), an automated or semi-automated format (e.g., ARCHITECT® and any successor platform) may have a relatively shorter incubation time (e.g., approximately 18 minutes for ARCHITECT®). Similarly, whereas a non-automated format such as an ELISA may incubate a detection antibody such as the conjugate reagent for a relatively longer incubation time (e.g., about 2 hours), an automated or semi-automated format (e.g., ARCHITECT® and any successor platform) may have a relatively shorter incubation time (e.g., approximately 4 minutes for the ARCHITECT® and any successor platform).

Other platforms which may be suitable include those available from Abbott Laboratories such as AxSYM®, IMx® (see, e.g., U.S. Pat. No. 5,294,404, which is hereby incorporated by reference in its entirety), PRISM®, EIA (bead), and Quantum™ II. Additionally, the assays, kits, and kit components can be employed in other formats, for example, on electrochemical or other hand-held or point-of-care assay systems. The present disclosure is, for example, applicable to the Abbott Point of Care (i-STAT®, Abbott Laboratories) electrochemical immunoassay system that performs sandwich immunoassays. Immunosensors and their methods of manufacture and operation in single-use test devices are described, for example, in U.S. Pat. No. 5,063,081, U.S. Pat. App. Pub. No. 2003/0170881, U.S. Pat. App. Pub. No. 2004/0018577, U.S. Pat. App. Pub. No. 2005/0054078, and U.S. Pat. App. Pub. No. 2006/0160164, which are incorporated herein in their entireties by reference for their teachings regarding same.

In one exemplary embodiment, with regard to the adaptation of an assay to the I-STAT® system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized capture antibody are adhered to a polymer coating of patterned polyvinyl alcohol over the electrode. This chip is assembled into an I-STAT® cartridge with a fluidics format suitable for immunoassay. On a portion of the wall of the sample-holding chamber of the cartridge there is a layer comprising the detection antibody labeled with alkaline phosphatase (or other label). Within the fluid pouch of the cartridge is an aqueous reagent that includes p-aminophenol phosphate.

In operation, a sample suspected of containing the biomarker is added to the holding chamber of the test cartridge and the cartridge is inserted into the I-STAT® reader. After the second antibody (detection antibody) has dissolved into the sample, a pump element within the cartridge forces the sample into a conduit containing the chip. Here it is oscillated to promote formation of the sandwich between the first capture antibody, the biomarker, and the labeled second detection antibody. In the penultimate step of the assay, fluid is forced out of the pouch and into the conduit to wash the sample off the chip and into a waste chamber. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of biomarker in the sample by means of an embedded algorithm and factory-determined calibration curve.

### Subjects

The exemplary methods described herein may be used on mammalian subjects who are experiencing or are expected to experience a prolonged period of physical inactivity. In one exemplary embodiment, the subject is a human subject. In one exemplary embodiment, the subject is an adult human subject. In one exemplary embodiment, the subject is an elderly human subject. The exemplary evaluation methods described herein may be used in connection with subjects who are involved in a clinical study where the subjects will experience a prolonged period of inactivity, for example, the subjects will be in bed for several days, such as 10 or more days. In one exemplary embodiment, the subject who is experiencing or is expected to experience a prolonged period of physical inactivity may be characterized as a subject in need of treatment (or “a subject in need thereof”) for the detrimental effects associated with a prolonged period of physical inactivity, for example, the development of fibrosis. The exemplary therapeutic and evaluation methods may be used in subjects who are experiencing or are expected to experience prolonged periods of inactivity in a hospital setting, a rehabilitation facility, a nursing home, a skilled nursing facility, or the subject's own home. For example, in certain embodiments, the subject may be a subject who may or will be restricted to bed due to an injury, illness, or surgery. In certain embodiments, the subject may be a subject who has self-imposed a prolonged period of inactivity, for example, a subject who is depressed.

### Control Values

In one exemplary embodiment, the difference between the levels of the one or more biomarkers in a test biological sample taken from the test subject 3 or more days after the subject becomes physically inactive are compared to a control value. The control value is based on the difference in the levels of the one or more biomarkers in comparable biological samples obtained from a control population, for example, the general population or a select population of human subjects who have experienced a comparable period of prolonged physical inactivity, for example, bed rest for 3-10 days or more. For example, the select population may be comprised of male subjects, or female subjects, or elderly subjects, and so forth. Accordingly, the control values selected may take into account the category into which the test subject falls (e.g., based on gender, age). Appropriate categories can be selected with no more than routine experimentation by those of ordinary skill in the art.

The difference and, therefore, the control value can take a variety of forms. For example, in certain embodiments, the control value can be the difference, either negative or positive, in mg/ml, ng/ml, and so forth of the circulating levels of the biomarker that is seen in untreated control subjects during a similar number of days of physical inactivity. The control value may be a percent change, either negative or positive, in the circulating levels of the biomarker in untreated control subjects during a comparable period of physical inactivity.

In certain embodiments, control values may be established by assaying a large sample of individuals in the general population or the select population and using a statistical model such as the predictive value method for selecting a positivity criterion or receiver operator characteristic curve that defines optimum specificity (highest true negative rate) and sensitivity (highest true positive rate) as described in Knapp, R. G., and Miller, M. C. (1992), Clinical Epidemiology and Biostatistics, William and Wilkins, Harual Publishing Co., Malvern, Pa., which is specifically incorporated herein by reference. In addition, reference intervals or expected values for the general population or the select population can be established by following the guidance from the Clinical and Laboratory Standards Institute (CLSI), document C28-A3c (2011).

### Interventions

Interventions that may be evaluated using the exemplary evaluation methods described herein include, but are not limited to, nutritional interventions. Examples of such nutritional interventions include, but are not limited to, high protein diets (e.g., >1 g protein/kg body weight/day, >1.2 g protein/kg body weight/day); supplements containing high doses of leucine (˜15 grams/day) or leucine metabolites such as HMB, alpha-ketoisocaproate (KIC) and alpha-hydroxyisocaproate (HICA); and high doses of essential amino acids (˜45 grams/day containing at least 15 grams of leucine).

### Systems for Evaluating the Efficacy of the Intervention

Exemplary systems for determining the efficacy of an intervention on the development of fibrosis associated with prolonged physical inactivity in a subject are also provided herein. In one exemplary embodiment, the system comprises one or more sub-systems for: (a) identifying a subject undergoing physical inactivity or expected to undergo physical inactivity in the near future; (b) taking a baseline biological sample from the subject identified in (a) before or on the day the subject becomes physically inactive; (c) administering the intervention to the subject; (d) taking a test biological sample from the subject identified in (a) 3 or more days after the subject becomes physically inactive; (e) measuring the levels of at least one biomarker selected from MMP-1, MMP-3, and MMP-10 in the biological samples obtained by the sub-systems of (b) and (d); (f) calculating the difference in the levels of the at least one biomarker in the biological samples obtained by the sub-systems of (b) and (d); (g) comparing the one or more differences calculated by sub-system (f) to a control value based on the difference in levels of the at least one biomarker in comparable biological samples taken from untreated control subjects at the beginning of and following a comparable period of physical inactivity; and (h) characterizing the intervention as efficacious if at least one of the following are true: (i) the difference for MMP-1 calculated by sub-system (f) is greater than the control value for MMP-1; (ii) the difference for MMP-3 calculated by sub-system (f) is smaller than the control value for MMP-3; and (iii) the difference for MMP-10 calculated by sub-system (f) is smaller than the control value for MMP-10.

In one exemplary embodiment, the sub-system for identifying a subject undergoing physical inactivity or expected to undergo physical inactivity in the near future identifies a subject scheduled to undergo a procedure that typically leads to a period of bed rest of three or more days. In one exemplary embodiment, the sub-system may also identify a subject who has an illness that typically leads to a period of bed rest of three or more days. Since elderly subjects typically undergo a longer period of inactivity following surgery or illness than younger subjects, the system may also comprise a sub-system that identifies the age of the subject. This latter sub-system may be the same as or different from the sub-system that identifies the status of the subject (i.e., if and when the subject may undergo a prolonged period of physical inactivity).

The exemplary systems may be used by facilities housing subjects who are undergoing, who may undergo, or who are expected to undergo a prolonged period of physical inactivity (e.g., bed rest) for an extended period of time, such as, for example, in a hospital, rehabilitation center, nursing home, and so forth. All of the sub-systems may be used directly by such facilities. Alternatively, some of the sub-systems may be used by testing facilities such as laboratories that report to or are directed to perform certain tests by the facility housing the subject. The exemplary systems may also be used by physicians directing the care of a subject who is undergoing, who may undergo, or who is expected to undergo a prolonged period of physical inactivity at a hospital, rehabilitation facility, nursing home, and so forth. The exemplary systems may also be used by companies developing interventions directed at reducing the development of fibrosis that is associated with a prolonged period of physical inactivity in human subjects.

### EXAMPLES

The exemplary methods and systems are based, at least in part, on inventors' discovery that levels of three (3) distinct circulating fibrosis biomarkers changed by statistically significant amounts in 18 healthy elderly subjects undergoing 10 days of bed rest. The exemplary methods and systems are also based, at least in part, on inventors' discovery that intervention with HMB altered the changes in levels of these circulating biomarkers.

Subjects

The following inclusion criteria were verified at screening: male or female ≧60 to ≦79 years of age; body mass index (BMI) ≧20 but ≦35; ambulatory with a Short Performance Physical Battery (SPPB) score of ≧9 (fully functional with no mobility limitations); and compliance with prescribed activity level. Exclusion criteria ruled out subjects who had undergone recent major surgery, had active malignancy (excepting basal or squamous cell skin carcinoma or carcinoma in situ of the uterine cervix); history of Deep Vein Thrombosis (DVT) or other hypercoagulation disorders; refractory anemia; history of diabetes or fasting blood glucose value >126 mg/dL; presence of partial or full artificial limb; kidney disease or serum creatinine >1.4 mg/dL; evidence of cardiovascular disease assessed during resting or exercise EKG; untreated hypothyroidism; liver disease; chronic or acute GI disease; uncontrolled severe diarrhea, nausea or vomiting; were actively pursuing weight loss; were enrolled in other clinical trials; could not refrain from smoking over the bed rest study period; or could not discontinue anticoagulant therapy over bed rest period. Potential subjects were also excluded if they were taking any medications known to affect protein metabolism (e.g., progestational agents, steroids, growth hormone, dronabinol, marijuana, HMB, free amino acid supplements, dietary supplements to aid weight loss).

The 24 healthy subjects initially involved in the study were randomized into two groups. Subjects in the treatment group received two β-hydroxy-β-methylbutyrate (HMB) sachets containing 1.5 grams of Ca-HMB (TSI, Salt Lake City, Utah), 4 grams of maltodextrin, and 200 milligrams of calcium with additional sweetener and flavoring agents. Subjects in the control group received two control sachets that were identical to the HMB sachets with the exclusion Ca-HMB. This study was a double-blinded study. Neither the investigators nor the subjects were informed of the identity of any of the study products during the clinical portion of the study. Subjects were instructed to consume a sachet twice daily by mixing a sachet into a non-caloric, non-caffeinated, non-carbonated, non-milk-based beverage of their choice. Treatment with HMB or Control was initiated 5 days prior to bed rest and was continued until the end of the rehabilitation period.

For diet stabilization over the pre-bed rest and bed rest periods, subjects were fed a metabolically controlled diet providing the RDA for protein intake (0.8 g protein/kg body weight per day). Total calorie needs were estimated using the Harris-Benedict equation for resting energy expenditure according to the following equation: For women=[655+(9.56×body weight in kg)+(1.85×height in cm)−(4.68×age in years)]×AF, and, For men=[66+(13.7×body weight in kg)+(5×height in cm)−(608×age in years]×AF, where AF=activity factor of 1.6 for the ambulatory period and 1.35 for the bed rest period. Given the total calorie and protein intakes, the remainder of the diet was manipulated to keep the non-protein calories at about 60% from carbohydrates and 40% from fat. Water was provided ad libitum.

After a diet stabilization of 5 days (ambulatory period), subjects remained in bed continuously for 10 days. While confined to bed rest, subjects were allowed to use the bedside commode for urination or were taken in a wheelchair for toileting. Subjects were given the option of taking a sponge bath or showering in a wheelchair. Prophylactic measures were taken to detect and prevent deep vein thrombosis including a blood d-dimer test followed by an ultrasound examination if d-dimer test was positive, passive range of motion exercise during bed rest, the use of TED hose and SCD over the bed rest period. Subjects were offered medication to help mitigate reflux problems associated with being supine. Subjects were constantly monitored by nursing staff and received a daily physical examination by the study physician.

Fasted blood samples were collected from subjects on Day 1 of bed rest and at the end of bed rest for measurement of biomarkers.

Subjects were exited from study if they permanently discontinued product during the pre-bed rest period (Day 1 to Day 5), or if they discontinued product during the bed rest period and had completed less than 8 days of bed rest. Subjects with a positive D-dimer test or ultrasound for deep vein thrombosis (DVT) diagnosis were also exited from the study.

A subject's outcome data were classified as unevaluable for the analysis if one or more of the following events occurred: A. Subject received wrong product, contrary to the randomization scheme; B. Subject received excluded concomitant treatment defined as medications or dietary supplements that affect weight or metabolism (e.g., progestational agents, steroids, growth hormone, dronabinol, marijuana, HMB, free amino acid supplements, dietary supplements to aid weight loss, and fish oil supplements); and C. Subject had <67% of total study product consumption at Final Visit/Exit as determined by product consumption records.

The final analytic sample size is n=18 subjects, n=8 in the control group (n=1 male, n=7 female) and n=10 (n=2 male, n=8 female) in the experimental HMB group.

Biomarker Analysis

Rules Based Medicine (Myriad RBM, Inc., Austin, Tex.) data generated from the RBM Human DiscoveryMAP v1.0 consists of n=187 biomarkers measured in serum collected at two time points, pre-bed rest and post-bed rest from 18 elderly subjects. The distribution of each marker was evaluated. Each marker has a least detectable dose (LDD) value, defined as the mean+3 standard deviations (SD) of 20 blank samples. For any subject whose marker result was ≦LDD, the LDD value as provided by RBM was imputed. If the marker result was >LDD, the original result was used. Any marker in which ≧30% of all subject's results were imputed was excluded from further statistical analyses. Of the initial n=187 RBM markers, n=63 markers from the RBM dataset were excluded from further analyses, leaving n=124 markers for evaluation.

Statistical Evaluation

Changes in RBM Biomarkers

The Control group was examined to see which fibrosis biomarkers changed over bed rest. Individual univariate dependent t-tests were performed on each of the 124 markers. There were a total of 8 participants who had matched data over bed rest within the Control group. From the initial univariate analysis, 3 fibrosis markers showed a statistically significant change over bed rest, with an unadjusted p-value less than 0.05.

Significance of ANCOVA Tests

In order to assess the changes in the markers over bed rest that may be mediated by HMB intervention, individual univariate ANCOVA analyses were subsequently performed on each of the 3 (unadjusted) significant fibrosis markers from the multiple dependent t-tests. Using a Bonferonni adjusted p-value of 0.0038 (0.05/13), three fibrosis markers were significant. This result indicates that these 3 fibrosis markers showed a statistical difference after bed rest between the Control and HMB groups while controlling for existing differences at baseline (pre-bed rest).

### Example 1

### Effect of HMB Intervention on Circulating Levels of MMP-3 in Subjects Who Have Experienced 10 Days of Bed Rest

Matrix metalloproteinase-3 (MMP-3), also known as Stromelysin-1, is an enzyme that is involved in digesting a number of extracellular matrix (ECM) molecules, as well as activating MMPs. The MMPs are centrally involved in morphogenesis, wound healing, tissue repair and remodeling in response to injury. A decrease in circulating levels of MMP-3 is associated with an increase in the development of fibrosis in human subjects.

As shown in Table 1, there was an average decrease of 2.80 ng/ml (or a 28% change) in circulating levels of MMP-3 in 8 control subjects after 10 days of bed rest. In contrast, the average decrease in MMP-3 levels in blood from 10 subjects treated with HMB was much less. As shown in Table 1, there was an as an average decrease of 2.71 ng/ml (or a 25% change) in blood levels of MMP-3 in the HMB treated subjects. These results show that HMB reduces or attenuates the decrease in blood levels of MMP-3 that occurs in untreated control subjects during prolonged bed rest.

### Example 2

### Effect of HMB Intervention on Circulating Levels of MMP-10 in Subjects Who Have Experienced 10 Days of Bed Rest

Matrix metalloproteinase-10 (MMP-10), also known as Stromelysin-2, is an enzyme that degrades proteoglycans and fibronectin. MMP-10 also is also involved in the breakdown of extracellular matrix in normal physiological processes, such as embryonic development, reproduction, and tissue remodeling. A decrease in circulating levels of MMP-10 is associated with the development of fibrosis in human subjects.

As shown in Table 1, there was an average decrease of 0.12 ng/ml (or an 18% decrease) in circulating levels of MMP-10 in 8 control subjects after 10 days of bed rest. In contrast, the average decrease in MMP-10 levels in blood from 10 HMB treated subjects was much less. As shown in Table 1, there was an average decrease of 0.09 ng/ml (or a 12% decrease) in blood levels of MMP-10 in the HMB treated subjects after 10 days of bed rest. These results show that HMB reduces or attenuates the decrease in blood levels of MMP-10 that occurs in untreated control subjects during prolonged bed rest.

### Example 3

### Effect of HMB Intervention on Circulating Levels of MMP-1 in Subjects Who Have Experienced 10 Days of Bed Rest

Matrix metalloproteinase-1 (MMP-1), also known as interstitial collagenase, is an enzyme that plays a significant role in the degradation of different types of collagen in extracellular matrix remodeling. MMP-1 is implicated in tissue remodeling and wound healing. Increased levels of MMP-1 may be needed for appropriate remodeling of damaged tissue (e.g., muscle tissue) induced by a prolonged period of physical inactivity.

As shown in Table 1, there was an average increase of 1.67 ng/ml (or a 24% increase) in circulating levels of MMP-1 in 8 control subjects after 10 days of bed rest. In contrast, there was a higher average increase of 2.17 ng/ml in circulating levels of MMP-1 in 10 HMB treated subjects after 10 days of bed rest. These values are significantly higher than the control values. These results show that HMB enhances the increase in blood levels of MMP-1 that occurs in untreated control subjects during prolonged bed rest.

CONCLUSION: Taken together all 3 markers indicate a protective effect of HMB against development of fibrosis during a prolonged period of physical inactivity in a human subject.

TABLE 1
Fibrosis Markers That Change With HMB Treatment Over Bed Rest

| | Control (n = 8) | | | | |
|---|---|---|---|---|---|
| | Pre-bed rest | | Post-bed rest | | |
| Biomarkers | Mean | Stdev | Mean | Stdev | Change |
| Matrix Metalloproteinase-1 | 10.38 | 7.51 | 12.05 | 8.23 | 1.67 ± 0.79 |
| (MMP-1) (ng/ml) | | | | | |
| Matrix Metalloproteinase-3 | 9.49 | 5.51 | 6.69 | 4.29 | −2.80 ± 1.25 |
| (MMP-3) (ng/ml) | | | | | |
| Matrix Metalloproteinase-10 | 0.56 | 0.26 | 0.44 | 0.18 | −0.12 ± 0.05 |
| (MMP-10) (ng/ml) | | | | | |
| | HMB (n = 10) | | | | |
| | Pre-bed rest | | Post-bed rest | | |
| Biomarkers | Mean | Stdev | Mean | Stdev | Change |
| Matrix Metalloproteinase-1 | 11.29 | 3.46 | 13.46 | 4.38 | 2.17 ± 1.06 |
| (MMP-1) (ng/ml) | | | | | |
| Matrix Metalloproteinase-3 | 10.90 | 1.16 | 8.19 | 1.17 | −2.71 ± 0.78 |
| (MMP-3) (ng/ml) | | | | | |
| Matrix Metalloproteinase-10 | 0.60 | 0.07 | 0.51 | 0.05 | −0.09 ± 0.04 |
| (MMP-10) (ng/ml) | | | | | |

* p-value was < 0.0001 for MMP-1 and MMP-10 and was equal to 0.0007 for MMP-3 as determined by univariate ANCOVA analysis.

What is claimed is:

1. A method of reducing the effect of prolonged physical inactivity on the development of fibrosis in a subject who is experiencing or is expected to experience prolonged physical inactivity in the near future, the method comprising: administering a therapeutically effective amount of β-hydroxy-β-methylbutyric acid (HMB) to the subject.

2. The method of claim 1, wherein the HMB is administered in a manner and for a time sufficient to elevate the increase in circulating levels of MMP-1 that occurs with prolonged physical inactivity in untreated control subjects.

3. The method of claim 1, wherein the HMB is administered in a manner and for a time sufficient to attenuate the decrease in circulating levels of MMP-3 that occurs with prolonged physical inactivity in untreated control subjects.

4. The method of claim 1, wherein the HMB is administered in a manner and for a time sufficient to attenuate the decrease in circulating levels of MMP-10 that occurs with prolonged physical inactivity in untreated control subjects.

5. The method of claim 1, wherein the HMB is administered in a manner and for a time sufficient to keep a test level of at least one of MMP-3 and MMP-10 in a test biological sample taken from the subject following 3 or more days of physical inactivity comparable to a baseline level of at least one of MMP-3 and MMP-10 in a baseline biological sample taken from the subject at the beginning of or prior to the prolonged physical inactivity.

6. The method of claim 1, wherein the subject is an elderly human subject.

7. A method of evaluating the efficacy of an intervention on the development of fibrosis in a subject who is undergoing or is expected to undergo a prolonged period of physical inactivity in the near future, the method comprising:
(a) measuring levels of at least one biomarker selected from MMP-1, MMP-3, and MMP-10 in a baseline biological sample taken from the subject before or on the day the subject becomes physically inactive;
(b) administering the intervention to the subject, wherein the intervention is first administered to the subject before or on the day the subject becomes physically inactive;
(c) measuring levels of the at least one biomarker measured in (a) in a test biological sample taken from the subject at 3 or more days after the subject has become physically inactive;
(d) calculating the difference between the levels of the at least one biomarker measured in the baseline biological sample and the at least one biomarker measured in the test biological sample;
(e) comparing the differences calculated in (d) to corresponding control values based on the difference between levels of the at least one biomarker in comparable biological samples taken from untreated control subjects at comparable time points; and
(f) determining that the intervention is efficacious when:
(i) the difference for MMP-1 calculated in (d) is greater than the control value for MMP-1;
(ii) the difference for MMP-3 calculated in (d) is smaller than the control value for MMP-3;
(iii) the difference for MMP-10 calculated in (d) is smaller than the control value for MMP-10; or
(iv) combinations of (i), (ii), and (iii).

8. The method of claim 7, wherein the subject is an elderly human subject.

9. The method of claim 7, wherein the baseline biological sample is a blood sample and the test biological sample is a blood sample.

10. The method of claim 7, wherein the prolonged physical inactivity is bed rest.

11. A method of evaluating the efficacy of an intervention on the development of fibrosis in a subject who is undergoing or is expected to undergo a prolonged period of physical inactivity in the near future, the method comprising:
(a) administering the intervention to the subject, wherein the intervention is first administered to the subject before or on the day the subject becomes physically inactive;
(b) measuring the level of at least one biomarker selected from MMP-1, MMP-3, and MMP-10 in a test biological sample taken from the subject 3 or more days after the subject becomes physically inactive;
(c) comparing the level measured in (b) to a baseline level of the at least one biomarker in a baseline biological sample taken from the subject before or on the day the subject becomes physically inactive; and
(d) determining that the intervention is efficacious when the measured level of the at least one biomarker in the test biological sample is comparable to the baseline level of the at least one biomarker in the baseline biological sample.

12. The method of claim 11, wherein the at least one biomarker consists of MMP-1.

13. The method of claim 11, wherein the at least one biomarker consists of MMP-3 and MMP-10.

14. The method of claim 11, wherein the subject is an elderly human subject.

15. The method of claim 11, wherein the intervention is a leucine metabolite selected from HMB, alpha-ketoisocaproate (KIC), alpha-hydroxyisocaproate (HICA), and combinations thereof.

16. The method of claim 11, wherein the intervention is a nutritional composition comprising a leucine metabolite selected from HMB, alpha-ketoisocaproate (KIC), alpha-hydroxyisocaproate (HICA), and combinations thereof.

17. A method of treating a subject at risk of developing fibrosis during a prolonged period of physical inactivity, the method comprising:
(a) determining whether at least one of the following is true:
(i) the MMP-3 level in a test biological sample of the subject taken during the prolonged period of physical inactivity is lower than the MMP-3 level in a baseline biological sample of the subject taken before or on the day the subject becomes physically inactive;
(ii) the MMP-10 level in a test biological sample of the subject taken during the prolonged period of physical inactivity is lower than the MMP-10 level in a baseline biological sample of the subject taken before or on the day the subject becomes physically inactive; and
(iii) the MMP-1 level in a test biological sample of the subject taken during the prolonged period of physical inactivity is within 20% of the MMP-1 level in a baseline biological sample of the subject taken before or on the day the subject becomes physically inactive; and
(b) administering to the subject a therapeutically effective amount of HMB when at least one of (i)-(iii) is true.

18. The method of claim 17, wherein the therapeutically effective amount of HMB is administered to the subject via a nutritional composition.

19. The method of claim 18, wherein the nutritional composition comprises at least one of: a source of protein, a source of carbohydrate, and a source of fat.

20. The method of claim 17, wherein the therapeutically effective amount of HMB is co-administered with alpha-ketoisocaproate (KIC), alpha-hydroxyisocaproate (HICA), or both.
